(19) United States
(12) Patent Application Publication
(10) Pub. No.: US 2017/0061822 A1
(43) Pub. Date: Mar. 2, 2017

(54) METHOD AND SYSTEM FOR PREPARING GUIDELINES FOR SELECTING FOODS AND OTHER SUBSTANCES TO ESTABLISH CELLULAR HEALTH

(71) Applicant: William J. McFaul, Jackson, NJ (US)

(72) Inventor: William J. McFaul, Jackson, NJ (US)

(21) Appl. No.: 15/245,993

(22) Filed: Aug. 24, 2016

Related U.S. Application Data
(60) Provisional application No. 62/211,422, filed on Aug. 28, 2015.

(51) Int. Cl. G09B 19/00; A61B 5/145; A61B 5/08; G06F 19/00

(52) U.S. Cl. CPC G09B 19/0092; G06F 19/3487; A61B 5/14546; A61B 5/082; A61B 2562/0285

(57) ABSTRACT

A method and system for selecting foods, medications and other substances that can enhance the nutrition required by cells to sustain life based on assessment of imbalances in an individual of cellular health related bodily substances required for cellular health are provided.

This patent application claims the benefit of priority from U.S. Provisional Application No. 62/211,422, filed Aug. 28, 2015, which is herein incorporated by reference in its entirety.

### FIELD OF INVENTION

The invention relates to the field of nutritional science for cells in the body. In particular, the invention relates to a system and method for selecting foods and other substances that can enhance the nutrition required by cells to sustain life based on an individual's personal needs to establish cellular health.

### BACKGROUND OF THE INVENTION

In terms of physiological health, a variety of substances have been designated as being nutritional substances or vitamins.

Biomedical research has identified the existence of various minerals and elements in the body that have antagonistic relationships such as sodium and potassium, calcium and magnesium, iron and manganese, bromine and iodine, cooper and zinc, carbon dioxide and oxygen, nitric oxide and oxygen, glutamate and glycine, albumin and creatinine, bilirubin and creatinine, dopamine and norepinephrine, DHA and EPA, DHA and DPA, NPY and PP, NPY and PYY, PREG and PROG, PREG and DHEA, as well as nitric oxide and hydrogen sulfide. Ratios between several of these substances have been disclosed while others including, but not limited to, calcium and zinc, nickel and zinc, glutamate and glycine, carbon dioxide and oxygen, nitric oxide and hydrogen sulfide, calnexin and calmodulin, CDKN1 and CDKN2, α-amylase and β-amylase, γ-amylase and α-amylase, norepinephrine and prolactin, α-carotene and β-carotene, lycopene and β-carotene, UPC1 and UPC2 still need to be determined.

Current technologies typically allow for assessment of minerals, elements and amino acids on the microscale level. These substances are oftentimes referred to as micronutrients and assumed to be essential for physiological health and well-being.

Despite the knowledge that minerals and elements interact, current diagnostic testing methodologies do not utilize these factors as a means to assess potential negative consequences on cellular health and well-being that enables them to perform their essential activities.

### SUMMARY OF THE INVENTION

The purpose of the present invention is to measure the level of cellular health related bodily substances in the body that have agonistic and antagonistic relationships that contribute to cellular abnormalities and to have these substances assayed using platforms that rely on tests based on nanoscale or lower levels of the substances. Levels of substances are determined through the collection of statistical data derived from testing of multiple individuals and incorporated into the database from which computations can establish standards modified on an ongoing basis as the volume of results increases. Imbalances in substances in an individual affecting cellular health can then be assessed by comparison to these standards and recommendations can be made for change in diet, medications, supplements, etc. to address the most effective means of correcting the imbalances.

Accordingly, an aspect of the present invention relates to a method for preparing guidelines for selecting foods and other substances to establish cellular health in an individual. In this method, a sample is obtained from an individual. Levels of cellular health related bodily substances in the sample indicative of cellular health are then assessed at nanomolecular (nanoscale) or lower levels. The assessed levels are then entered into a computer and compared via the computer to a panel of cellular health related bodily substance levels from a control group. Any imbalances in the assessed levels are reported and guidelines for selecting foods and other substances to correct the imbalances are provided.

Another aspect of the present invention relates to a system for preparing guidelines for selecting foods and other substances to establish cellular health in an individual. The system comprises a means for assessing nanomolecular (nanoscale) or lower levels of cellular health related bodily substances in the sample indicative of cellular health in a sample obtained from an individual. In one nonlimiting embodiment, this means comprises a nanoplatform analyzer. The system further comprises a computer which compares the assessed levels of cellular health related bodily substances in the sample indicative of cellular health to a panel of cellular health related bodily substances from a control group and a report generated by the computer of any imbalances in the assessed levels and guidelines for selecting foods and other substances to correct the imbalances.

### DETAILED DESCRIPTION OF THE INVENTION

A large number of minerals, metals, elements and amino acids in the body interact through agonistic and antagonistic activities. These interactions between minerals and elements, or the combination of amino acids in conjunction with minerals and elements, are part of the formation of signaling molecules that form pathways of signaling activity that regulate cellular activity. Imbalances in ratios between any of the constituents of the signaling molecules will prevent cells from having the ability to adequately connect and interact in order to regulate mechanisms required for the maintenance of psychological and physiological health. Accordingly, imbalances between these interactions can create inadequate cellular health.

The present invention provides a method and system to assess levels of cellular health related bodily substances inclusive of, but not limited to minerals and elements as well as amino acids, hormones, bacteria, viruses and fungi in an individual to identify interactions and imbalances and to compare results from testing by the use of a computer (e.g. a smartphone platform) to assess needs for cellular health and well-being in the individual. In the present invention, ratios of antagonistic relationships between cellular health related bodily substances measured at nanoscale levels or lower in a sample obtained from an individual are incorporated into a panel that reports imbalances between such ratios as a means of enabling individuals or clinical professionals to access personal levels in order to change dietary habits, as well as assess the need for medicines, nutritional supplements, etc. to restore imbalances that may exist.

The term “level” as used herein is meant to be inclusive of ratios of selected cellular health related bodily substances.

Testing to establish ratios can be performed using samples of hair, saliva, blood, plasma, serum, urine, fecal matter, diagnostic imaging, breath analysis, skin tissue sampling or the use of devices to assess cellular health related bodily substances within cells or within extracellular matter.

These assessments utilize nanoplatform analyzers or may use ones that function in scales lower even than nano (i.e. 10⁻⁹), e.g. pico (10⁻¹²), femto (10⁻¹⁵) or atto (10⁻¹⁸). Technology currently exists for nanoscale assessments in conjunction with smart phone technology via companies such as Theranos using blood samples (see theranos with the extension .com of the world wide web) and Nanobiosym using saliva (see nanobiosym with the extension .com of the world wide web). Such technologies can be routinely adapted in accordance with the present invention to assay interactions and imbalances between cellular health related bodily substances necessary to maintain cellular health and wellbeing.

Imbalances are recognized by statistical deviations based on each cellular health related bodily substance, or ratios of selected cellular health related bodily substances, as established as being consistent with current diagnostic testing standards.

Based upon these imbalances, the present invention then provides a report and guidelines for modifying these imbalances through diet, medications, supplements or other means for the purpose of maintaining cellular health and well-being. Detection of imbalances in accordance with the present invention also allows for assessment of potential downstream abnormalities that can disrupt cellular health. Nonlimiting example of antagonistic relationships for which ratios are assessed in accordance with the present invention include excessive calcium displacing magnesium and zinc while diminished levels of zinc impacts interactions with selenium within cells; excessive levels of BRCA3 creates imbalances between BRCA1 and BRCA2; and excessive levels of nitric oxide creates imbalances between oxygen and carbon dioxide. Additional examples include, but are in no way limited to, excessive nitric oxide depleting hydrogen sulfide, excessive nickel depleting zinc, excessive norepinephrine depleting deplete prolactin, and excessive calnexin depleting calmodulin.

As a nonlimiting example, upon assessment of an imbalance between gasotransmitters such as nitric oxide and hydrogen sulfide in an individual in accordance with the method and system of the present invention, a guideline to modify dietary intake to include nuts that contain nitric oxide and to decrease intake of cruciferous vegetables that contain hydrogen sulfide may be provided.

What is claimed is:

1. A method for preparing guidelines for selecting foods and other substances to establish cellular health in an individual, said method comprising:
(a) obtaining a sample from an individual;
(b) assessing cellular health related bodily substance levels in the sample indicative of cellular health at nanomolecular (nanoscale) or lower levels;
(c) entering the assessed levels into a computer;
(d) comparing via the computer the assessed levels to a panel of cellular health related bodily substance levels from a control group;
(e) reporting any imbalances in the assessed levels; and
(f) providing guidelines for selecting foods and other substances to correct the imbalances.

2. The method of claim 1 wherein cellular health related bodily substance levels are assessed via a nanoplatform analyzer.

3. The method of claim 1 wherein the sample is selected from the group consisting of hair, saliva, blood, plasma, serum, urine, fecal matter, breath analysis, a diagnostic image and skin tissue.

4. The method of claim 1 wherein the cellular health related bodily substances assessed are selected from the group consisting of minerals, elements, amino acids, hormones, bacteria, viruses and fungi.

5. A system for preparing guidelines for selecting foods and other substances to establish cellular health in an individual, said system comprising:
(a) a means for assessing nanomolecular (nanoscale) or lower levels of cellular health related bodily substance levels indicative of cellular health in a sample obtained from an individual;
(b) a computer which compares the assessed levels of cellular health related bodily substances to a panel of cellular health related bodily substances from a control group; and
(c) a report generated by the computer of any imbalances in the assessed levels and guidelines for selecting foods and other substances to correct the imbalances.

6. The system of claim 5 wherein said means for assessing nanomolecular (nanoscale) or lower levels of cellular health related bodily substances indicative of cellular health in a sample comprises a nanoplatform analyzer.

7. The system of claim 5 wherein the sample is selected from the group consisting of hair, saliva, blood, plasma, serum, urine, fecal matter, breath analysis, a diagnostic image and skin tissue.

8. The system of claim 5 wherein the cellular health related bodily substances assessed are selected from the group consisting of minerals, elements, amino acids, hormones, bacteria, viruses and fungi.
